(19) United States
(12) Patent Application Publication
(10) Pub. No.: US 2020/0375649 A1
(43) Pub. Date: Dec. 3, 2020

(54) DEVICE AND METHOD FOR COOLING LIVING TISSUE

(71) Applicants: RecensMedical.Inc, Ulsan (KR); UNIST (ULSAN NATIONAL INSTITUTE OF SCIENCE AND TECHNOLOGY), ULSAN (KR)

(72) Inventor: Gun-Ho KIM, Ulsan (KR)

(73) Assignees: RecensMedical.Inc, Ulsan (KR); UNIST (ULSAN NATIONAL INSTITUTE OF SCIENCE AND TECHNOLOGY), ULSAN (KR)

(21) Appl. No.: 16/993,229

(22) Filed: Aug. 13, 2020

Related U.S. Application Data
(63) Continuation of application No. 15/828,449, filed on Dec. 1, 2017.

(51) Int. Cl. A61B 18/02; A61F 9/00; A61F 9/007; A61F 7/00

(52) U.S. Cl. CPC A61B 18/02; A61F 9/0008; A61F 9/00736; A61B 5/4821; A61F 9/007; A61M 2210/0612; A61F 7/007

(57) ABSTRACT

A device for cooling a living tissue is disclosed. The cooling device solves severe patient's waiting and clinic work load due to time-consuming anesthesia required by conventional therapies. The cooling device also significantly reduces psychological burden and pain of the patient for ocular anesthesia. The cooling device includes a cooling unit configured to cool a target area and a heat dissipating unit configured to dissipate heat from the cooling unit. The heat dissipating unit is also configured to regulate internal air flow to increase heat dissipating efficiency.

### CROSS-REFERENCE TO RELATED APPLICATION

This application is a continuation of U.S. patent application Ser. No. 15/828,449, filed on Dec. 1, 2017, the contents of which are all hereby incorporated by reference herein in their entirety.

### BACKGROUND OF THE DISCLOSURE

### Field

The present disclosure relates to a device and methods of rapid anesthesia or analgesia through rapid cooling using a thermoelectric element based on Peltier effect, which is in direct contact with mucosal skin or living tissue of an eye.

### Background

With the aging population and increasing number of patients with diabetes, vision threatening retinal diseases such as age-related macular degeneration, diabetic retinopathy, and diabetic vein occlusions are increasing rapidly. For the last decade, intravitreal injection therapy (IVT), the periodic injections of medication such as ranibizumab and aflibercept directly into the patient eyes, has been found to be more successful in treating the aforementioned vision threatening retinal diseases than laser therapy and vitreous replacement procedures, and become the standard of care in these patients. As a result, more than 90% of patients with age-related macular degeneration, diabetic retinopathy, and retinal vein occlusions are treated with IVTs, and according to the American Society of Retina Specialists, the number of IVTs is estimated to be over 6 million in 2016 in the United States alone and reach at least 10 million by 2020.

IVT is a painful and psychologically stressful procedure, and patients often demand maximal anesthesia before an injection. Retina specialists typically choose one among three anesthesia methods when such maximal anesthesia is required, cotton tipped applicators soaked with lidocaine, viscous anesthetic, or subconjunctival lidocaine injection. These methods require several minutes for the onset of maximal anesthesia, increasing the time required for patient preparation by several fold. While the method of eye drops of topical anesthetics is the most time-efficient method, the level of anesthesia is moderate and patients often complain of injection pain.

Both maximal and moderate anesthesia options rely on chemical anesthesia agents. Compared with anesthesiology in other areas, ophthalmic anesthesia requires several unique carefulness such as systemic diseases of the patient, systemic reaction by treated medicines and interaction between such medicines and anesthetic agent, which has significant effects even on the success of the ophthalmic surgery. In addition to the possible side effects, chemical anesthesia agents often result in adverse effects when applied to the eye surface such as eye dryness and soreness, which further lead to patient discomfort.

The rapidly increasing number of IVTs has resulted in severe strain in ophthalmic clinic work flow and long patient waiting time, forcing retina specialists to sacrifice patient experience for managing their busy clinics. The trade-offs between the quality and time efficiency of current ocular anesthesia methods as well as the several adverse effects and medical complications of ocular anesthetic agents indicate unmet needs for a non-invasive and time-efficient method for maximal anesthesia.

### SUMMARY OF THE DISCLOSURE

The present disclosure or teaching is contemplated to solve the problem in the conventional art, and thus an object of the present disclosure is to provide cooling device and method enabling precise manipulation thereof.

Another object of the present disclosure is to provide the cooling device and method preventing contamination or contagion through use thereof.

Still another object of present disclosure is to provide the cooling device and method allowing stable operation constantly with performing rapid anesthesia.

According to one embodiment of the present disclosure, a device for cooling a living tissue may comprises a body; a first end portion disposed in the body, the first end portion provided with a cooling medium configured to thermally coupled with a cooling tip to cool a target area; and a second end portion disposed in the body and formed opposite to the first end portion, wherein a center of mass portion of the device is located within the first end portion and a grip for facilitating manipulation of the device is disposed at a predetermined position of the first end portion.

Here, the grip may be disposed corresponding to the center of mass portion, and the center of mass portion may be configured to be located between 50% and 90% of a length of the device from an end of the second end portion. The grip may be disposed corresponding to the center of mass portion, and may be located within the center of mass portion, that is the range of 50% to 90% of a length of the device from an end of the second end portion.

Further, a stopper may be formed at the grip adjacent to the first end portion and may protrude from the grip to guide a gripping position of a user. The grip may include a seating groove formed at a portion of the grip with which a finger of a user is in contact and recessed into the body by a predetermined depth.

Further, the center of mass may be provided with the cooling medium, a cooling unit, and a heat dissipation unit. More specifically, the cooling medium may extend outside the first end portion by a predetermined length, the cooling unit may include a thermoelectric element configured to cool the cooling medium, and the heat dissipation unit may be configured to dissipate heat generated from the cooling unit.

In addition, the device may further comprise a container disposed between the cooling medium and the heat dissipation unit and defining a seating space for the thermoelectric element. The container may include a guide portion having a shape corresponding to an outer shape of the cooling medium, and the cooling medium may be inserted and seated in the guide portion by a predetermined depth.

Further, a battery may be disposed opposite to the cooling medium and the cooling tip with reference to the center of mass portion. 30% of a total weight of the device may be concentrated on the center of mass portion, when a battery is disposed at the second end.

According to another embodiment of the present disclosure, a device for cooling a living tissue may comprise a body; a first end portion disposed in the body, the first end portion including a cooling unit provided with the cooling medium that is configured to cool a target area, and a heat dissipation unit dissipating heat from the cooling unit, and a second end portion disposed in the body and formed opposite to the first end portion, wherein the heat dissipation unit includes a fan generating active air flow at fins provided at the heat dissipation unit, and an inlet introducing external air and an outlet discharging air in the device are formed at predetermined regions in the first end portion in which the fan is disposed, respectively, and wherein the air flows in and flows out within a predetermined portion of the body.

Here, the body may have air flow in which passive air flow proceeding opposite to a direction of gravity is combined with the active air flow caused by the fan, while the device is inclined by a predetermined angle with regard to ground to be operated by a user.

In addition, the device may further comprise a partition configured to block air flow between the first end portion including the cooling unit that cools the target area and the second end portion including a controller that controls the cooling unit. The inlet may be disposed in front of the heat dissipation unit, and the outlet may be disposed in a rear of the fan.

Air flow discharged from the outlet may include first air flow discharged directly from the fan and second air flow reflected from the partition.

Further, at least one of the inlet and the outlet may be formed at a tapered portion of the body. The inlet may be oriented upwardly inclined toward the stopper or the grip, and the outlet may be oriented downwardly inclined toward the second end portion.

Here, Air flow between the inlet and the outlet may pass through fins of a heat sink disposed within the body, and a time period for passing the fins may be different from positions where the air is introduced through the inlet.

A clearance between the fin of the heat dissipation unit and a case of the body may be set within a predetermined range. A controller for the fan may be disposed within the second end portion to control introducing external air from the inlet and discharging the external air to the outlet. Air flow may not be oriented horizontal at the inlet and the outlet.

According to still another embodiment of the present disclosure, a device for cooling a living tissue may comprise a removable cooling tip configured to contact a target area in the living tissue to cool the target area; and a tip case configured to support the cooling tip, the tip case including a coupling member configured to the cooling tip to the device, wherein the cooling tip is configured to be thermally and electric-potentially coupled with a cooling medium provided in the device.

The cooling tip may have an electric potential corresponding to an electric potential of any one of the cooling medium and a thermoelectric element in contact with the cooling medium by electric-potential coupling. At least one of the cooling tip and the cooling member is electric-potentially coupled with an element that can store electric charge.

Further, the tip case may have an electric circuit configured to determine a reuse of the cooling tip. The electric circuit may be configured to be opened by an electrical manner using an electric signal transmitted to the electric circuit or by a physical manner using force applied to the electric circuit.

In addition, the device may further comprise a reuse alarm unit for signaling the reuse of the cooling tip, and the reuse alarm unit may be configured to use any one of display, sound, or vibration to inform a user of the reuse of the cooling tip.

Here, the device may further comprise a reuse determination unit configured to determine whether the cooling tip is reused by determining an electric connection at the electric circuit; and a reuse prevention unit configured to limit use of the device using a predetermined manner when it is determined that the cooling tip is reused. The device may further comprise a circuit opening unit configured to open the electric circuit for preventing the reuse of the cooling tip, when it is determined that the cooling tip is in the normal use.

Further, the coupling member of the tip case may adopt one of a hook lock mechanism and a screw mechanism. The device may further comprise an elastic member providing elastic force to the cooling member in a direction opposite to a coupling direction of the tip case, to thermally couple the cooling member with the cooling tip. A grip with a predetermined shape may be formed at the tip case to facilitate detaching of the tip case.

Details of embodiments will be described in the following with reference to the accompanying drawings. Other features will be apparent from the description and drawings, and from the claims.

### BRIEF DESCRIPTION OF THE DRAWINGS

The present disclosure will become more fully understood from the detailed description given herein below and the accompanying drawings, which are given by illustration only, and thus are not intended to limit the scope of the present application, and wherein:

FIGS. 1A-1H are views showing an overall configuration and individual configurations of a cooling device according to an embodiment of the present disclosure;

FIGS. 2A-2F are views showing a configuration for dissipating heat source produced in the cooling device according to the embodiment of the present disclosure; and

FIGS. 3A-3G are views showing a configuration for the tip case of the cooling device according to the embodiment of the present disclosure.

### DETAILED DESCRIPTION

Description will now be given in detail according to exemplary embodiments disclosed herein, with reference to the accompanying drawings. For the sake of brief description with reference to the drawings, the same or equivalent components may be provided with the same reference numbers, and description thereof will not be repeated. In general, a term such as “module” and “unit” may be used to refer to elements or components. Use of such a term herein is merely intended to facilitate description of the specification, and the term itself is not intended to give any special meaning or function. In the present disclosure, that which is well-known to one of ordinary skill in the relevant art has generally been omitted for the sake of brevity. The accompanying drawings are used to help easily understand various technical features and it should be understood that the embodiments presented herein are not limited by the accompanying drawings. As such, the present disclosure should be construed to extend to any alterations, equivalents and substitutes in addition to those which are particularly set out in the accompanying drawings.

It will be understood that although the terms such as first, second and the like may be used herein to describe various elements, these elements should not be limited by these terms. These terms are generally only used to distinguish one element from another.

It will be understood that when an element is referred to as being “connected with” or “coupled with” another element, the element can be directly connected with the other element or intervening elements may also be present. In contrast, when an element is referred to as being “directly connected with” or “directly coupled with” another element, there are no intervening elements present.

A singular representation may include a plural representation unless it represents a definitely different meaning from the context.

Terms such as “comprise”, “include” or “have” are used herein and should be understood that they are intended to indicate an existence of several components, functions or steps, disclosed in the specification, and it is also understood that greater or fewer components, functions, or steps may likewise be utilized. Moreover, due to the same reasons, it is also understood that the present invention includes a combination of features, numerals, steps, operations, components, parts and the like partially omitted from the related or involved features, numerals, steps, operations, components and parts described using the aforementioned terms unless deviating from the intentions of the disclosed original invention.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the disclosure in use or operation in addition to the orientation depicted in the figures. For example, if any element in the figures is turned over, elements described as “below” or “beneath” other elements or features would then be oriented “above” the other elements or features. Thus, the example term “below” can encompass both an orientation of above and below. Such an element may be otherwise oriented (rotated 90 degrees or at other orientations) and the spatially relative descriptors used herein interpreted accordingly.

The cooling device according to the present disclosure may be configured to deliver rapid and safe anesthesia to an ocular surface to assist an injection into an eye or other medical therapies for the eye. Although the cooling device and method according to the embodiment of the present disclosure are described with regard to ocular treatment, such device and the method may be applicable to devices and methods for cooling other subjects.

Configuration of Cooling Device

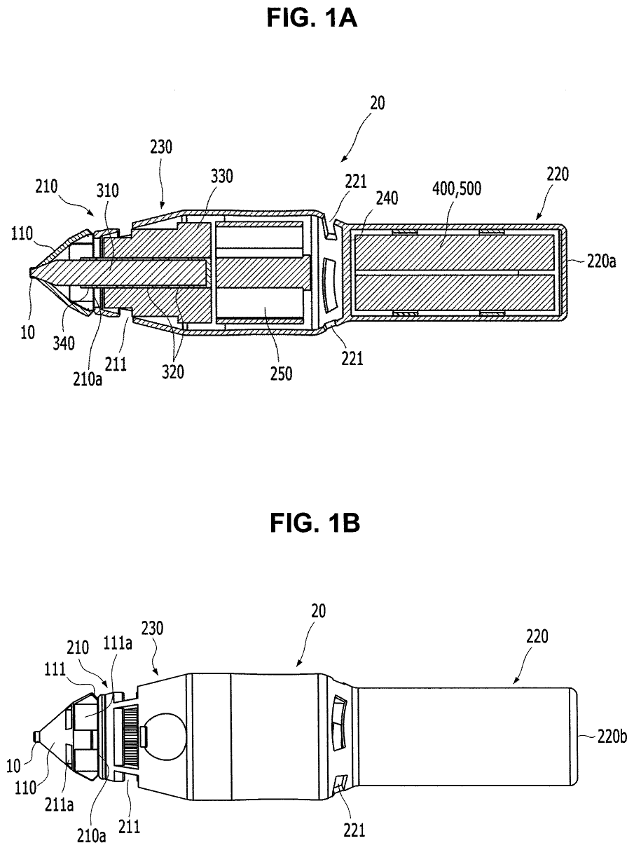

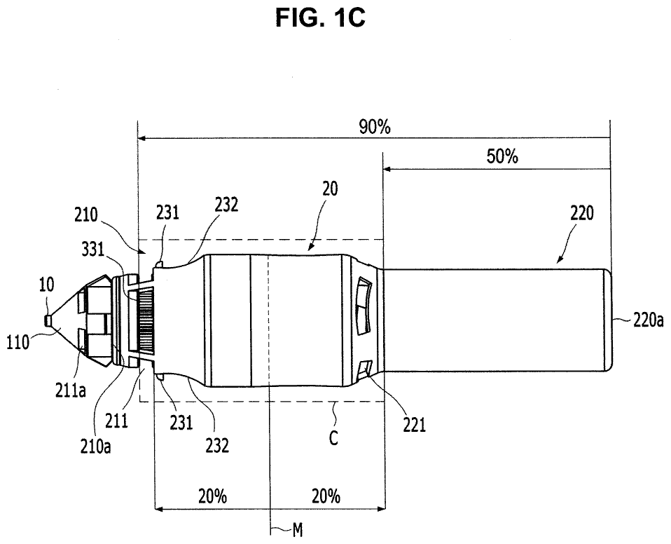

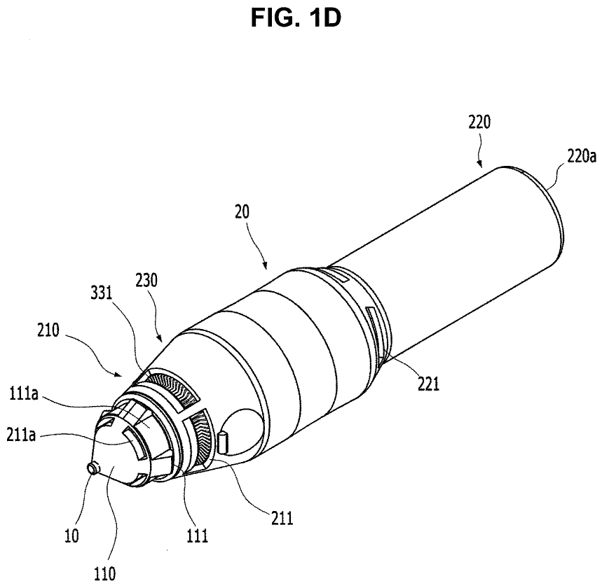

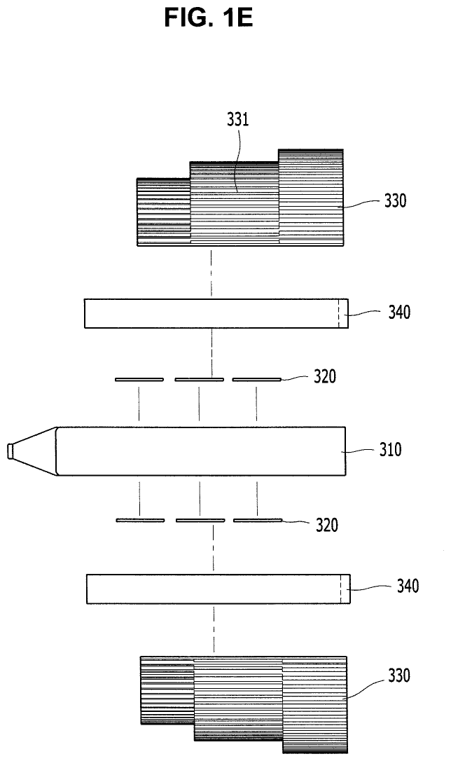

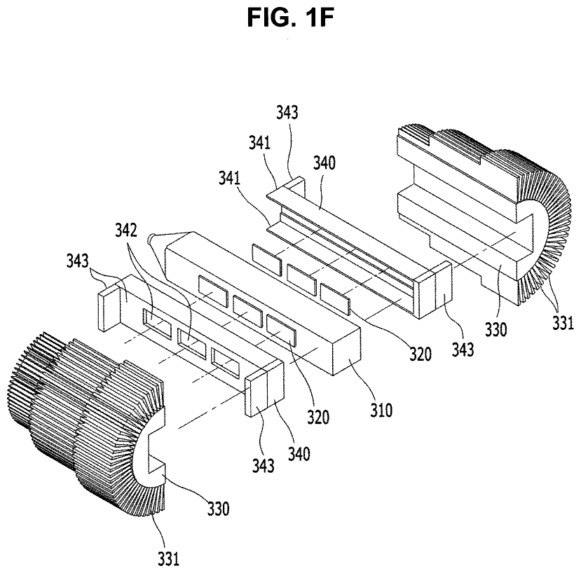

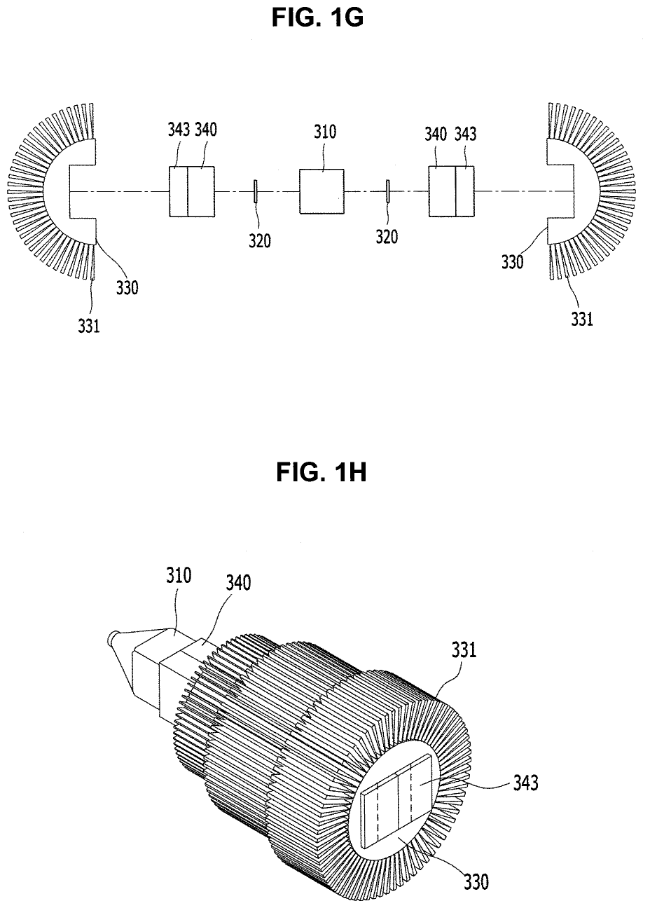

FIGS. 1A-1H are views showing an overall configuration and individual configurations of a cooling device according to an embodiment of the present disclosure.

FIGS. 1A and 1B are views showing the overall configuration according to the embodiment of the present disclosure.

Referring to FIG. 1A, the cooling device according to the embodiment of the present disclosure may comprise a body 20 including a first end portion 210 provided with a cooling medium 310 thermally coupled with a removable cooling tip 10 that directly contacts with a target area to be cooled and a second end portion 220 formed at an end opposite to the first end portion 210. The target area may be a portion of a surface of any tissue to be anesthetized by cooling and may be simply referred to as a target hereinafter for brevity

A center of mass portion C (see FIG. 10) of the body 20 (or the cooling device) may be generally located relatively adjacent to or within the first end portion 210 and may include a grip 230 for facilitating manipulation of the cooling device that is disposed at a predetermined position of the first end portion 210. Since the body 20 may occupy most of the cooling device as shown in the related drawings of the present disclosure, the center of mass portion C of the body 20 may be considered a center of mass portion of the entire cooling device. The body 20 (or the cooling device) may include a first end 210a facing or adjacent to the target that is the ocular surface to be cooled and anesthetized and a second end 220a disposed opposite to the first end 210a. Thus, the first end portion 210 may include the first end 210a and a portion of the body 20 that is adjacent to the first end 210a. Particularly, the first end portion 210 may extend from the first end 210a toward the second end 220a by a predetermined length. Likewise, the second end portion 220 may include the second end 220a and a portion of the body 20 which is adjacent to the second end 220a and may extend from the second end 220a toward the first end 210a by a predetermined length. Further, in view of a relative position with regard to the target, the first end 210a and the first end portion 210 may be regarded as a proximal end and a proximal end portion, as the first end 210a and the first end portion 210 are relatively adjacent to the target. In the same manner, the second end 220a and the second end portion 220 may be regarded as a distal end and a distal end portion.

The cooling medium 310 may be provided on one end of the removable cooling tip 10. The cooling medium 310 may physically contact the removable cooling tip 10 and may extend to the second end portion 220. Further, the cooling medium 310 may be in physical contact with a thermoelectric element 320. The cooling medium 310 may comprises thermally conductive material. In some embodiments, thermal conductivity may accompany electrical conductivity of the cooling medium 310. Such a cooling medium 310 may be referred to as a cooling rod, a cooling arm, and so forth. Further, the cooling tip 10 may be detachably coupled to the cooling device, more specifically a tip case 110 for a hygienic reason. The cooling tip 10 may be exposed from an end of the cooling device to contact the target. The cooling tip 10 may be made of thermally conductive material to facilitate transferring of cooling power from the cooling medium 310 to the target. In some embodiments, due to the thermal conductivity, the cooling tip 10 may also have an electrical conductivity. More specifically, the cooling tip 10 may comprise a body which has a thin wall and defines a space configured to receive a portion of the cooling medium 310, more specifically an end or a tip thereof so as to be in physical or thermal contact with such a portion. For example, the cooling tip 10 may comprise a cap or receptacle member.

A heat sink 330 may be configured to dissipate heat generated by the thermoelectric cooling unit. A fan 250 may be configured to forcibly discharge the heat dissipated from the heat sink 330 to an outside of the body 20 or the cooling device. The thermoelectric element 320 and the heat sink 330 may be radially disposed in an inner space of the body 20 about the cooling medium 310.

Meanwhile, in view of the relative position with regard to the target or the patient's eye, the cooling device may be classified into a front portion adjacent to the patent's eye while the cooling device is in use, and a rear portion relatively disposed apart from the patent's eye. In such classification, the front portion may include the heat sink 330, the cooling medium 310, a disposable and/or removable tip 10 and the like, and the rear portion may include a power source 400 and a controller 500. The controller 500 may comprise a printed circuit board and a processor installed on the printed circuit board along with other electric components. The controller 500 may be electrically connected to other components of the cooling device as shown and described herein to control operation thereof. Therefore, any features relating to the operation of the cooling device should be regarded as the features of the controller 500 as well.

According to the principle of the present disclosure, the cooling device may have the feature of being portable. The portability may imply that the user may easily and precisely manipulate the cooling device using the hand. For this purpose, the grip 230 may be placed in a front portion (i.e., the first end portion 210) like a writing tool (a pencil or a ballpoint pen) to naturally induce and support a stable grip. Further, mass or weight of components located in the rear portion (i.e., the second end portion 220) may be configured to be less than 50% of total device mass or weight. The target cooling function, which is another important feature of the cooling device, may be based on heat absorption and dissipation occurring on two opposite surfaces of the thermoelectric element (a Peltier element) 320, respectively. The heat sink 330 may be in contact with a heat dissipating or emitting surface of the thermoelectric element 320 and a heat absorbing or cooling surface thereof may be in contact with the cooling medium 310. Then, an interface for cooling the target, i.e., the cooling tip 10 may come into contact with cooling medium 310. Therefore, the cooling power may be transferred to target to be cooled subsequently through the thermoelectric element 320, the cooling medium 310, and the cooling tip 10, which are thermally and physically coupled to each other. More specifically, the thermoelectric element 320 generally may have a low coefficient of performance and may fail to provide sufficient cooling power flux to maintain tissue at a temperature relevant for anesthesia, if the thermoelectric element 320 is directly placed on the tissue. As described above, the cooling device may adopt the cooling medium 310 that collects the cooling power of multiple (or single) thermoelectric elements 320 and may concentrate this cooling over a small area via the cooling tip 10. Therefore, the cooling device may produce a sufficient cooling power flux required for rapid and sustainable low temperature for cooling of tissue.

If a distance between the cooling tip 10 and the thermoelectric element 320, that is, a length of the cooling medium 310, is relatively long, loss of the cooling power may occur in terms of heat transfer. For this reason, a cooling engine (i.e., the cooling medium 310 and the thermoelectric element 320) should be located close to the cooling tip 10. For such proximity to the cooling tip 10, the cooling medium 310, the thermoelectric element 320 and the heat sink 330 may be all located in the first end portion 210, i.e., the front portion. Therefore, the first end portion 210 where the grip 230 is located may naturally include the center of mass portion C since most of the components are concentrated in the first end portion 210.

Referring to FIG. 1B together with FIG. 10, the grip 230 may be provided at a location within a range from a center of mass M of the cooling device to 30% of an overall length of the cooling device toward the first end portion 210. The grip 230 may be located within the center of mass portion C of the cooling device so that the user may easily manipulate the device. In some specific embodiments, the grip 230 may be provided at a location within a range of 60% to 90% of the overall length of the cooling device from the second end 220a of the body, i.e., a rear end of the body 20 or the cooling device.

Meanwhile, in the embodiment of the present disclosure, the cooling device including the body 20 may be elongated for a predetermined length and may be formed in a shape such as a pencil which the user easily holds and handles. A shape of a cross-section of the body 20 may be circular, oval, polygonal, or a combination thereof.

As described above, the removable cooling tip 10 in direct contact with the target area may be installed at one end, i.e., at a front end of the cooling device. The first end portion 210 may further extend and thus include such an end of the cooling device to which the cooling tip 10 is attached together with the tip case 110.

Further, the first end portion 210 may be directed toward the target for anesthesia and the second end portion 220 may face upward from the ground at a predetermined angle, while the cooling device is in use.

The grip 230 may be disposed adjacent to the first end portion 210 and may be formed at a position where the user can easily hold and manipulate the cooling device. A stopper 231 may be formed at a boundary or an interface of the grip 230 adjacent to the first end 210a. The stopper 231 may protrude by a predetermined height and may guide gripping by the user.

The stopper 231 may serve to fix a position of the user's finger within the grip 230 to be close to the first end 210a. Such configuration of the stopper 231 may stably maintain the gripping of the user at the time of sophisticated work such as medical treatment or the like.

The stopper 231 may extend above from an outer surface of the body 20, and a shape thereof may be variously embodied. It is preferable that the position and shape of the stopper 231 may be determined in accordance with a shape and a position of a seating groove 232 formed so as to correspond to a shape of the finger.

The grip 230 may be formed with the seating groove 232 recessed into the body 20 to have a shape mating with the finger that is used to hold the body 20. One or more seating grooves 232 may be formed on the outer surface of the body 20, and may be shaped to have the same contour as the finger that is inserted into the seating grooves 232 for holding the cooling device.

For example, when the body 20 comprises an elongated rod such as the writing tool, a plurality of grips 230 may be disposed on the outer surface of the body 20 to receive the thumb, the index finger and the middle finger with having shapes corresponding to touching surfaces of these fingers. With such a configuration, the grips 230 may maintain the stable gripping of the cooling device.

Meanwhile, the grip 230 may be detachably installed in order for the user to selectively adjust a position of the grip 230 on the body 20.

FIG. 10 is a view showing a configuration of the center of mass portion, and FIG. 1D is a view showing a configuration of the grip according to the embodiment of the present disclosure.

According to the present disclosure, the center of mass portion C may include the center of mass M of the cooling device. More specifically, the center of mass portion C may comprise a portion of the cooling device between 50% and 90% of the overall device length from a rear end of the device, i.e., the second end 220a. Alternatively, the center of mass portion C may comprise a portion extending from the center of mass M toward the front end (i.e., the first end 210a or the first end portion 210) and the rear end (i.e., the second end 220a or the second end portion 220) by 20% of the overall device length, respectively. Under the influence of the gravity, the center of mass M and the center of mass portion C may be center of gravity and a center of gravity portion, respectively.

The grip 230 may be located within the center of mass portion C to be associated with the center of mass M in the cooling device, such that the user may perform the precise manipulation easily.

According to the preferred embodiment of the present disclosure, the center of mass M may be configured to be located between 40% and 80% of the overall device length from the second end 220a of the body, for easily gripping and handling the cooling device by the user. Further, the grip 230 may be located within a portion extending from the center of mass M toward the front end (i.e., the first end 210a or the first end portion 210) by 30% of the overall device length. In the preferred embodiment, the grip 230 may be provided within the center of mass portion C or may be partially overlapped with the center of mass portion C so that the user may precisely manipulate the device.

With such configuration, more than 30% of the total mass or weight of the cooling device may be concentrated on the center of mass portion C such that the user may precisely handle the device while gripping, and stability of the gripping may be continuously maintained.

In another embodiment of the present disclosure, the center of mass portion C may be formed over a range of the 55% and 95% of the overall device length from the second end 220a of the body 20. The grip 230 may be disposed within such a center of mass portion C. Alternatively, the grip 230 may be disposed outside of the center of mass portion C for allowing the ergonomic gripping.

The user may grip the body by a hand as if using the writing tool. That is, the grip 230 may be held using the thumb, the index finger, and the middle finger. Further, the user may operate the device within a range of approximately 45 degrees in both lateral directions (for example, left and right directions) with reference to an axis vertical to a target surface in the patient's eye.

Meanwhile, one or more stoppers 231 may be formed at the interface or the end of the grip 230 adjacent to the first end 210a or first end portion 210 and may protrude by a predetermined height. The stopper 231 may prevent the finger received in the grip 230 from moving toward the removable cooling tip 10 and interfering with the removable cooling tip 10, and may also serve to maintain the stable gripping on the body

Further, the grip 230 may include the seating groove 232 provided on the outer surface of the body 20. The seating groove 232 may be recessed into the body 20 and may have the shape mating with the finger that is used to hold the body 20. Further, in order to guide the gripping, a pair of stoppers 231 which functions as barriers may be oppositely provided on the outer surface of the body 20 to protrude therefrom by 3-10 mm. Therefore, the finger may be unable to move over the stoppers toward the end of the cooling device such that user may stably grip the cooling device. The stopper 231 may also cause friction with the fingers which enables tight holding of the cooling device. Therefore, the user may easily detach the tip case 110 with holding the body 20. Moreover, the stopper 231 may support the fingers against or opposite to the direction of the force applied to the target treated by the cooling tip 10, such that the cooling tip 10 may be in thermal contact with the target stably.

FIGS. 1D-1H are views showing a configuration of a thermoelectric cooling unit according to the embodiment of the present disclosure.

Referring to FIGS. 1D-1H along with FIGS. 1A and 10, the center of mass portion C may be provided with the cooling medium 310 extending within the body 20 toward the first end 210a of the first end portion 210 or the front end of the cooling device by a predetermined length.

The cooling medium 310 may further extend outside the first end portion 210 from the body 20 by a predetermined length. The thermoelectric cooling unit may include the cooling medium 310 and a thermoelectric element 320 configured to cool the cooling medium 310. The heat sink 330 may dissipate the heat generated by the thermoelectric cooling unit. Such thermoelectric cooling unit and the heat sink 330 may be disposed within the center of mass portion C. Further, the fan 250 may be disposed adjacent to the thermoelectric cooling unit, more specifically in a rear of the thermoelectric cooling unit, and may form a heat dissipation unit for cooling the thermoelectric cooling unit together with the heat sink 330. In some embodiments, the fan 250 may have a case with a cylinder shape, an axis of which is parallel with the length of the cooling device, such that the shape of the fan 250 is in the accordance with the circular or elliptical cross-section of the cooling device. Further, the fan 250 may comprise an axial flow fan, the axis of which is configured to be parallel with or coincide with the central axis of the cooling device, to facilitate air flow along such a central axis. As clearly shown in FIGS. 1A and 10, the thermoelectric cooling unit and the heat dissipation unit as defined above may be disposed within the center of mass portion C. Alternatively, the thermoelectric cooling unit may be defined as including the thermoelectric element 320 only, and the heat dissipation unit may be also defined as including the heat sink 330 only.

The thermoelectric element 320 may refer to an element that generates the Peltier effect in which the heat is absorbed on one side of two different conductors and the heat is emitted on the opposite side thereof according to a direction of current when direct current is applied to both sides of two different conductors. In general, the thermoelectric element 320 may be also referred to as the Peltier element.

More specifically, the Peltier effect is reverse to the Seebeck effect and refers to the generation of temperature difference by simultaneously producing heat emission and heat absorption when potential difference is applied to the element including two different conductors. The heat absorbing surface of the thermoelectric element 320, i.e., the cooling surface thereof may be thermally coupled with the cooling medium 310. Thus, the cooling power of the thermoelectric element 320 may be transferred to the cooling medium 310 through surface contact. In other words, the heat may be transferred from the cooling medium 310 to thermoelectric element 320 through the surfaces thereof in contact with each other to achieve a low temperature of the cooling medium 310.

The heat generating or emitting surface of the thermoelectric element 320 may radiate or dissipate the heat through a plurality of cooling fins 331 formed at the heat sink 330, using thermal coupling with the heat sink 330.

The thermal and mechanical/physical coupling may be achieved in order of the cooling medium 310, the cooling surface of the thermoelectric element 320, the heat emitting surface of the thermoelectric element 320, and the heat sink 330. Due to such coupling, more than 30% of the total mass or weight of the cooling device may be concentrated on the center of mass portion C.

Meanwhile, as shown in FIGS. 1A and 10, the second end portion 220 may be provided with a power source 400 for supplying power and a controller 500 for controlling the operation of the cooling device. The power source 400 may comprise a battery, an electric storage, or similar devices. The controller 500 and the power source 400 may be spatially and thermally isolated or shielded from the components installed in the center of mass portion C, using a partition 240. When the power source 400 is disposed at the second end portion 220 as described above, the center of mass portion C in the first end portion 210 may have more than 30% of the total mass or weight of the cooling device, relatively.

On the other hand, the mass or weight of the second end portion 220 may be configured to be less than 50% of the total mass or weight of the cooling device.

The cooling medium 310 may be interposed between the thermoelectric element 320 and the removable cooling tip 10 so as to transfer the cooling power of the thermoelectric cooling unit 320 to the removable cooling tip 10. The cooling medium 310 may physically contact both of the thermoelectric element 320 and the removable cooling tip 10.

More specifically, the cooling medium 310 may be thermally coupled to one end of the removable cooling tip 10 while physically contacting with such an end of the removable cooling tip 10, and may extend to the second end portion 220 from such a contacting end. The cooling medium 310 may comprises a body having various shapes of cross section such as a rectangular or circular shape. The embodiment of the present disclosure will be described with reference to the cooling medium 310 with the body extending by a predetermined length and having a rectangular cross section. Such a cooling medium 310 may be referred to as a cooling rod, cooling column, a cooling arm, and the like.

The cooling (or heat absorbing) surface of the thermoelectric element 320 may physically contact an outer surface of the cooling medium 310 to transfer the cooling power to the cooling medium 310, and the heat emitting surface of the thermoelectric element 320 may physically contact the an outer surface of the heat sink 330.

The single thermoelectric element 320 and the single heat sink 330 may be applied to provide the cooling power to the cooling medium 310. However, for more rapid cooling and anesthetization, a pair of thermoelectric elements 320 and a pair of heat sinks 330 may be provided to be symmetrically positioned with respect to the longitudinal direction of the cooling medium 310. More pairs of the thermoelectric elements 320 and the heat sink 330 may be installed for the same reason. More specifically, the pair of thermoelectric elements 320 may be radially disposed about the cooling medium 310, being in contact therewith. Further, the pair of heat sinks 330 may enclose the cooling medium 310 and the thermoelectric elements 320 while directly contacting the thermoelectric elements 320 only.

The container 340 may be interposed between the cooling medium 310 and the heat sink 330. The thermoelectric element 320 may be disposed within the container 340 while contacting the cooling medium 310 and the heat sink 330. The container 340 may have a shape and a structure corresponding to shapes and structures of the thermoelectric element 320 and the heat sink 330 such that the thermoelectric element 320 and the heat sink 330 may be combined as an assembly using the container 340. The container 340 may be made of material with the low thermal conductivity and thus may thermally isolate the cooling medium 310 from the heat sink 330.

The pair of containers 340 may be symmetrically disposed with regard to the cooling medium 310 and may have complementary structures to be coupled with each other. When such a pair of container 340 is coupled to each other, the thermoelectric elements 320 and the heat sink 330 may be placed on a predetermined position where the thermoelectric elements 320 directly contact the cooling member 310 and the heat sinks 330 also directly contact the thermoelectric elements 320, respectively to be thermally and physically coupled with one another.

Meanwhile, the container 340 may include a first guide 341 for cooling medium 310. The first guide 341 comprises a rib or a flange formed on a surface of the container 340 facing the cooling medium 310 and extending toward the cooling medium 310. The first guide 341 may be configured to support the cooling medium 310 by contacting a surface thereof, and thus may guide the cooling medium 310 to be located at a predetermined portion within the container 340. Further, a plurality of first guide 341 may be provided to the container 340 for more rigid support. In this case, the first guides 341 of the container 340 may enclose sides of the cooling medium 310 and may relatively define a recess into which at least a portion of the cooling medium 310 may be inserted. The first guide 341 may have substantially the same length as the facing surface of the cooling medium 310. Further, such a first guide 341 may be provided to each of the containers 340. Therefore, when the pair of containers 340 are coupled to each other, the first guides 341 of these containers 340 may define a space receiving a body of the cooling medium 310. That is, the containers 340 as the assembly may enclose the cooling medium 310. Therefore, with such guides 341, the containers 340 may securely hold the cooling medium 310.

Further, the container 340 may include a second guide 342 for the thermoelectric element 320. The second guide 342 may comprise a recess or an opening formed on a surface of the container 340 facing the cooling medium 310 to receive the thermoelectric element 320. The second guide 342 may be shaped to correspond to an outer shape of the thermoelectric elements 320 and thus may immovably receive the thermoelectric element 320. Therefore, the thermoelectric element 320 may be stably inserted and seated in the second guide 342 while exposing two opposite heat absorbing and emitting surfaces to the cooling medium 310 and the heat sink 330 from the container 340.

Further, the container 340 may include the third guide 343 in which the heat sink 330 is received. The third guide 343 may comprise a recess formed on a surface of the container facing the heat sink 330. The third guide 343 may receive a portion of the heat sink so as not to be separated from the container 340. Alternatively, the third guide 343 may comprise a flange protruding from the surface of the container 340 facing the heat sink 330. More specifically, the flange may be provided at an edge of the container 340. The flange may be latched on a side of the heat sink 330 and thus the heat sink 330 may be stably placed on the container 340. For more stable placement, a plurality of flanges (i.e., the third guide) may be formed on the container 340 to be latched on the different sides of the heat sink 330. In this case, the recess may be defined as the third guide 341 by the plurality of flanges spaced apart from each other.

With the configuration as described above, the thermoelectric element 320 and the heat sink 330 may be firmly held by the container 340 and may stably maintain the physical contact with the cooling medium 310. More specifically, the cooling or heat absorbing surface of the thermoelectric element 320 may be in contact with the cooling medium 310, and then, the heat sink 330 may be in contact with the heat dissipating or emitting surface of the thermoelectric element 320.

Air Flow in Cooling Device

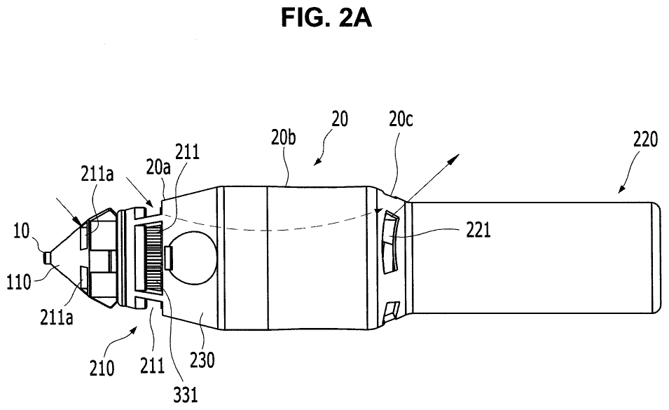

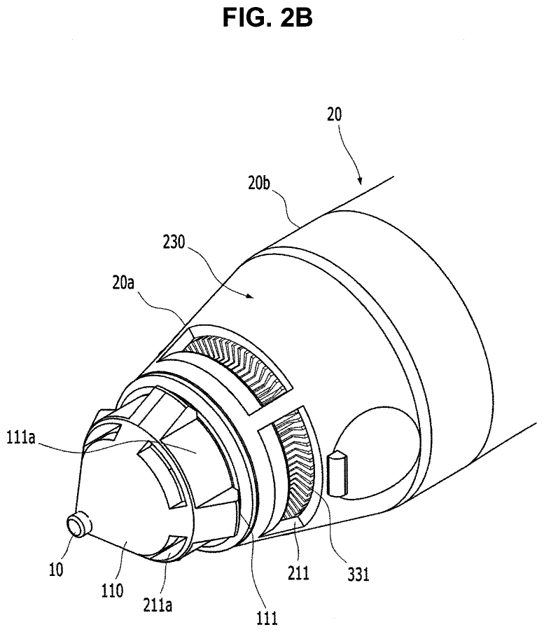

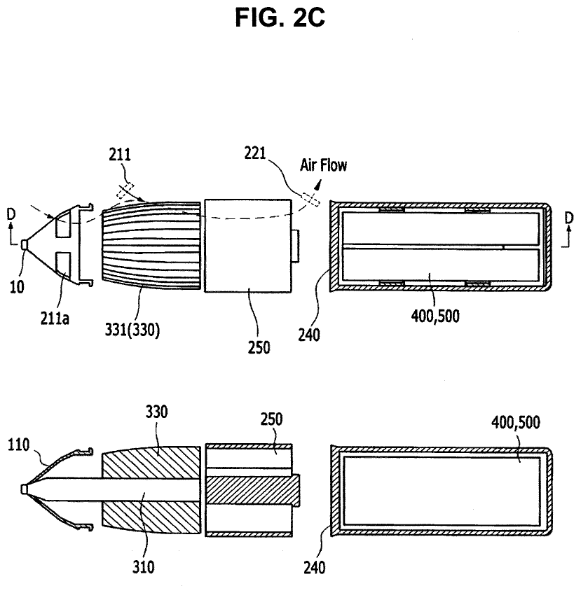

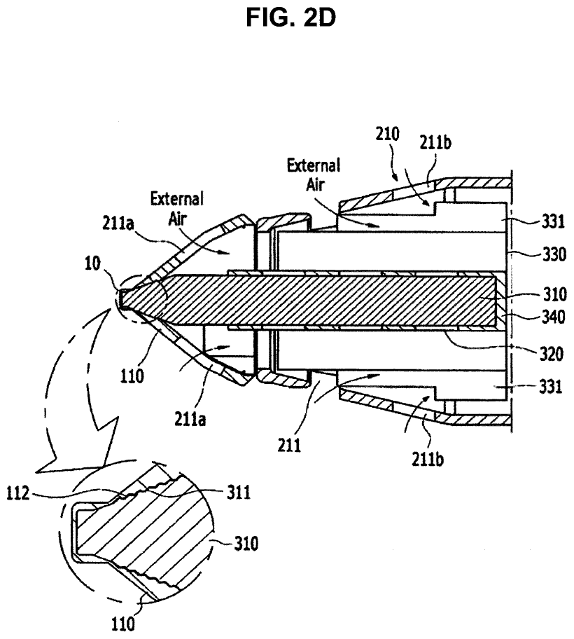

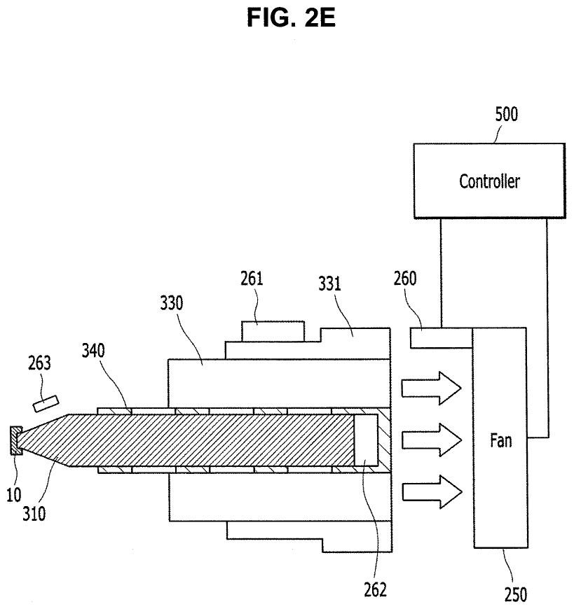

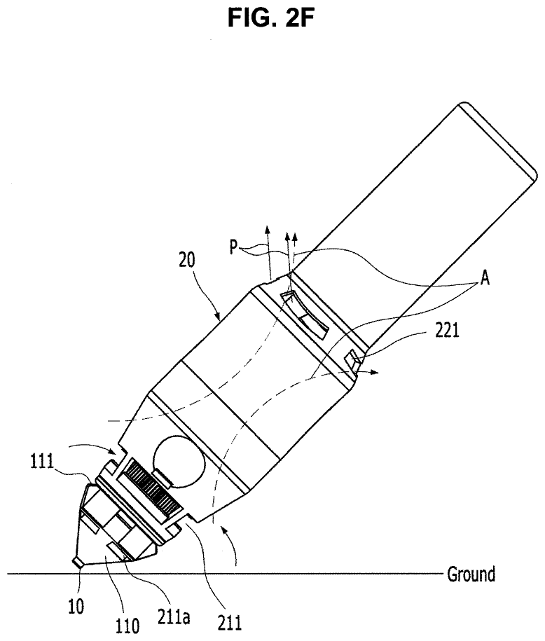

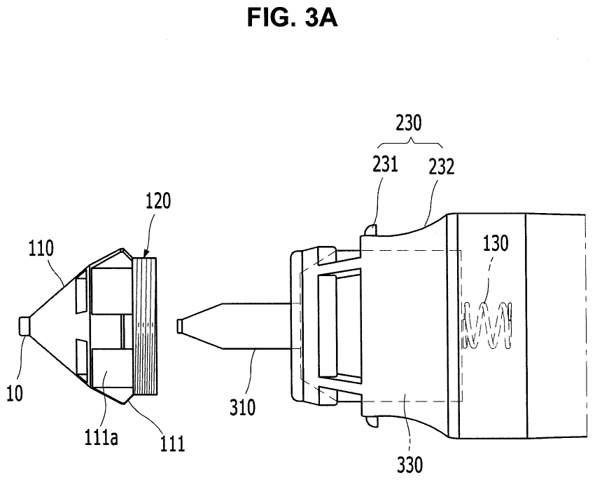

FIGS. 2A-2F are views showing a configuration for dissipating heat source produced in the cooling device according to the embodiment of the present disclosure. Particularly, FIGS. 2A and 2B are views showing the air flow and the configuration therefor according to the embodiment of the present disclosure.

Referring to FIG. 2A, the tip case 110 may be located at the front portion of the cooling device. The tip case 110 may be configured to be disposable and removable. The tips case 110 may be further configured to control the air flow within the body 20 of the cooling device. More specifically, the tip case 110 may include a sub inlet 211a provided on the body thereof. Such a sub inlet 211a may comprise a circular shape, an oval shape, a slit, and so on. In addition, the fan 250 (see FIG. 1A) may be disposed within the body 20 and an outlet 221 may be provided behind the fan 250. Therefore, heated air in the body 20 may be discharged through the fan 250 which is configured to control the air flow from the front portion to the rear portion of the cooling device.

Meanwhile, an inlet 211 may be formed in front of the heat sink 330 having the fin 331 of the heat dissipation unit, and the outlet 221 may be formed in a rear of the fan 250 of the heat dissipation unit. The inlet 211 and the outlet 221 may be provided within the first end portion 210. The inlet 211 may serve to introduce external air into the body 20, and the outlet 221 may serve to dissipate the air heated in the body 20 to the outside of the cooling device.

Meanwhile, the inlet 211 and the outlet 221 may be formed at tapered portions, i.e., inclined portions of the body 20.

In an aspect of the outer shape or contour, the cooling device may comprise a first region 20a that is inclined outwardly from the first end 210a that is coupled to the tip case 110 to a predetermined position of the first end portion 210. Further, the cooling device may comprise a second region 20b that is generally horizontal, i.e., parallel to a center axis with extending from the first region 20a to a predetermined position of the center of mass portion C. That is, the center of mass portion C and the center of mass M may be included in the second region 20b.

Further, the cooling device may have a third region 20c that is inclined inwardly from an end of the second region 20b to a predetermined position of the first end portion 210. The third region 20c may be located corresponding or adjacent to the center of mass portion C. In such a configuration, the inlet 211 may be located at the first region 20a that is an inclined portion of the first end portion 210. The inlet 211 may comprise a plurality of through holes having a predetermined diameter. In contrast, the outlet 221 may be located at the third region 20c that is inclined in an opposite direction to the first region 20a. More specifically, due to the disposition in the first and second regions 20a and 20b, the inlet 211 may be oriented to be inclined upwardly (i.e., outwardly) and the outlet 221 may be oriented to be inclined downwardly (i.e., inwardly).

In addition, as shown in FIGS. 2A and 2B, the inlets 211 may be elongated slots extending in a circumferential direction of the body 20, and may be arranged along the circumferential direction to be spaced apart from one another. Alternatively, each of the inlets 211 may have a ring shape, and may also have various shapes other than describe above. Further, the same configuration for the inlet 211 may be applied to the outlet 221. In some embodiments, however, the shape of the inlets 211 may not be elongated in a circumferential direction, but the array of the inlets 211 may be made in a circumferential direction of the body 20, which has a shape elongated in a circumferential direction of the body 20.

As the inlet 211 and the outlet 221 may be disposed on the first and third regions 20a and 20c that are inclined, respectively, this may be advantageous for easily introducing the external air to the heat sink 330 and effectively discharging the heated air from the fan 250 to the outside of the body 20 or the cooling device.

The inlet 211 may be disposed adjacent to a front end of the grip 230 to guide the external air into the body 20. The outlet 221 may be spaced apart from the inlet 211 by a predetermined distance that covers the heat sink 330 and the fan 250. The shape of the inlet 211 and the outlet 221 may be a plurality of circular holes, a long hole, or various shapes. In detail, the inlet 211 may be provided between the grip 230 and the first end 220a, i.e. the removable tip case 110, and the outlet 221 may be provided at an interface between the first end portion 210 and the second end portion 220, such that air flow is not hindered by user grip. More specifically, the outlet 221 may be provided at an interface between the grip 230 and the second end portion 220, such that air flow is not hindered by user grip.

Generally, the cooling device may be manipulated with a predetermined angle with regard to the ground or a contact surface (i.e., target in the eye). In this case, since a temperature of the heated air from the heat sink 330 may be higher than a surrounding air temperature, the heated air may move upward, i.e. in the direction opposite to the gravity or in a direction vertical to the ground and may exit outside through the outlet 221. Further, the external air may flow in via the inlet 211 and the sub inlet 211a due to a pressure difference in the body. With this procedure, the heat sink 230 inclined as suggested in the present disclosure may be cooled by natural convection even with no additional forced air flow.

FIGS. 2C and 2D are views showing a configuration for an internal air flow according to the embodiment of the present disclosure.

Referring to FIGS. 2C and 2D, an air flow path for cooling between the inlet 211 and the outlet 221 may be configured to pass through a plurality of fins 331 formed on an outer surface of the heat sink 330 in the body 20.

The air flow within the cooling device may have significant influence on the heat absorption and dissipation of the heat sink 330. The heat absorption may occur between the heat emitting surface of the thermoelectric element 320 and the heat sink 330, and the heat dissipation may occur between the heat sink 330 and the air flow in the body 20. A direction or an orientation of the air flow may determine the effective heat dissipation by causing the air flow to pass through every fin 331 on the heat sink 330.

In some embodiments, the effective dissipation may be related to a speed of air flow passing between the fins 331 of the heat sink 330. In view of the speed of the air flow, the air introduced through the inlet 211 may flow parallel to tops or bottoms of the fins 331 through a space between such fins 331 when there is no control for guiding the air flow. That is, the introduced air may flow parallel to a surface extending between the bottoms of the adjacent fins 331. However, if the air flow is directed to the bottoms of the fins 331, such air flow may gradually spread out and may travel toward the tops of the fins 331 due to the reduced speed thereof by the frictional resistance at the bottoms, while passing through the space between the fins 331, and thus may extensively contact surfaces of the fins 331 to enhance the efficiency of the heat dissipation. For these reasons, the inlet 211 may be configured to direct the air flow to the bottoms of the fins 331. More specifically, the inlet 211 may be located at a position that is able to guide the air flow to the bottoms of the fins 331. Further, the inlet 211 may be oriented toward the fins 331, more specifically the bottoms of the fins 331. That is, the inlet 211 may face the bottoms of fins 331 located at an entrance portion or a front portion of the heat sink 330. In some implementations, the air flow may be directed above the bottom of the fin 331 or below the bottom of the fin 331 by 2 mm.

Further, the outlet 221 may be designed such that a maximum volume of air may escape by minimizing the resistance interrupting the air flow. Generally, the air flow discharged from the fan 250 may travel and hit a wall of the body and this may create a back flow in the opposite direction. In this case, turbulent flow may be created in the rear of the fan 250 and may act as additional resistance to the discharge of the air flow. Therefore, it is required for the cooling device to directly guide the air flow discharged from the fan 250 to the outside of the cooling device. For this reasons, the outlet 221 may be oriented toward an outlet portion of the fan 250 discharging the air, such that the discharged air is smoothly directed toward and directly passes through the outlet 221.

As described above, the inlet 211 and the outlet 221 may be configured to be oriented toward an entrance of fins 331 (or the heat sink 330) and an exit of the fan 250, respectively. For such a configuration, the inlet 211 and outlet 221 may be located at the first and third regions 20a and 20b that are inclined to face the fins 331 and the fan 250, respectively. Therefore, the air flow may be formed in a smoothly curved path from the inlet 211 to the outlet 221. For these reasons, the air flow may extensively contact the surfaces of the fins 331 and may directly travel out of the cooling device through the outlet 221. Further, as the sub inlet 211a may initially introduce the air to the front end of the heat sink 330, this may facilitate an extended smoother path of the air flow. Moreover, as the partition 240 is located adjacent to the outlet 221, the partition 240 may also guide the air flow in the device toward the outlet 221. In this case, the air flow discharged from the outlet 221 may include first air flow discharged directly from the fan 250 and second air flow reflected and guided by the partition 240.

Referring to FIG. 2D, the cooling device may further include an additional inlet 211b formed at the body 20. In view of the inlet 211 and the sub inlet 211a, the additional inlet 211b may be considered a third inlet. This third inlet 211b may have the same configuration and function as first and second inlets, i.e., the inlet and the sub inlet 211 and 211a. More specifically, the third inlet 211b may be located to overlap with the heat sink 330, particularly, the fins 331 thereof. That is, the third inlet 211b may be located above the heat sink 330. Due to the overlapping position of the third inlet 211b, a time period for which the air flow through the third inlet 211b stays in the body 20 as well as a distance by which the air flow through the third inlet 211b travels in the body 20 may be different from those of the first and second inlets 211 and 211a. Therefore, with such different time periods and distances of the first to third inlets 211, 211a, 211b, the air flows via the first to third inlets 211, 211a, 211b may contact the entire body of heat sink 330 more uniformly, and the heat exchange between the heat sink 330 and the air flow may be facilitated.

FIG. 2D also shows the configuration of the heat sink 330 for facilitating the air flow in the body 20 according to the embodiment of the present disclosure. An outer shape of the heat sink 330 may correspond to the shape of the body 20 surrounding the heat sink 330. More specifically, a contour or an outline of the heat sink 330 may correspond to a contour of an inner surface of the body 20 facing the heat sink 330. Further, the fins 331 of the heat sink 330 may be spaced apart from the inner surface of the body 20 by a predetermined distance. Specifically, the predetermined distance between the outline of the heat sink 330 and the inner surface of the body 20 may be smaller than 3 mm. In another embodiment, the predetermined distance between the outline of the heat sink 330 and the inner surface of the body 20 may be smaller than 10% of the length of the heat sink 330. Meanwhile, the cooling device may further comprise a fan control unit for controlling the fan 250 to forcibly introduce the external air via the inlets 211, 211a, and 211b and then discharging such air flow via the outlet 221. Meanwhile, the air flow discharged via the outlet 221 may include air flow generated from the fan 250 and air flow reflected from the partition 240. Further, the outer surface of the fan 250 may conform to the inner surface of the body 20, and therefore may guide airflow in the cooling device to flow through the fan 250. In some embodiments, the difference between the outer diameter of the body 20 and the outer surface of the fan may be less than 10 mm, in order to maximize the diameter of the fan 250 and hence air flow rate.

FIG. 2E is a view showing a configuration of the controller related to the heat dissipation according to the embodiment of the present disclosure.

According to some embodiments of the present disclosure, the cooling device may comprise the controller 500 configured to control the fan 250 which introduces the external air at the inlets 211, 211a, and 211b and forcibly discharges the heated air to the outlet 221. The controller 500 may control a motor for driving the fan 250 and a thermal sensor 260 provided in the body 20 and detecting a temperature in the body 20. As the fan 250 discharges the heated air with a high temperature, the thermal sensor 260 may be disposed adjacent to the outlet portion of the fan 250, i.e. in the rear of the fan 250. Such thermal sensor 260 may detect the temperature of heated air that may flow around within the body 20 and directly affect the operation of the nearby components. Thus, locating the thermal sensor 260 in the rear of the fan 250 may be advantageous for maintaining the stable operation of the cooling device by controlling the internal temperature.

When the temperature detected by the thermal sensor 260 is higher than a predetermined temperature, the controller 250 may selectively operate the fan 250 to maintain the temperature in the body 20 below the predetermined temperature.

A display unit may be further installed on the outer surface of the body 20 to indicate the temperature detected by the thermal sensor 260. The user may determine whether the cooling device may be used, based on change in temperature and color indicated in the display unit.

Further, thermal sensors 261 and 262 may be additionally provided to the heat sink 330 and the cooling medium 310, respectively. With these thermal sensors 261 and 262, temperatures of heat sink 330 and the cooling medium 310 may be monitored and properly regulated by the controller 500. The temperatures detected by the thermal sensors 261 and 262 may be also indicated in the display unit such that the user may manually interrupt the operation of the cooling device under an abnormal condition.

Meanwhile, a thermal sensor 263 may be further installed on the removable cooling tip 10 to detect a temperature of the removable cooling tip 10. The current temperature of the removable cooling tip 10 may also be indicated on the display unit. More specifically, the current temperature of the removable cooling tip 10 may be displayed by numbers or color change. Therefore, the user may determine whether the cooling tip 10 is prepared for cooling and anesthetizing the target based on the information provided by the display unit and may also interrupt the anesthetization of the target based thereon. To easily detect the temperature of the cooling tip 10, a remote sensor such as an infrared temperature sensor may be adapted as the thermal sensor 263 for the cooling tip 10. The cooling tip 10 may be configured to cause black body radiation by coating for correct detection by the remote thermal sensor 263.

As described above, the cooling device may include the first end portion 210 provided with the removable cooling tip 10 and the cooling medium 310 thermally coupled with the removable cooling tip 10. The cooling device may further include the fan 250 generating the air flow around the heat sink 330 that may be configured to dissipate the heat of the thermoelectric element 220. The air flow inside and outside the cooling device may be formed around the first end portion 210. The inlet 211 and the outlet 221 may be formed at a predetermined portion of the cooling device in which the fan 250 is disposed.

FIG. 2F is a view showing the air flow during the use or operation of the cooling device according the embodiment of the present disclosure.

While the cooling device is in use or operation, i.e. the user causes the cooling device to contact the target to be anesthetized by cooling, the cooling device may be actually in a posture having a predetermined angle with regard to the ground, as shown in FIG. 2F. In such a posture, the cooling device may have internal air flow that is a combination of passive air flow P proceeding opposite to the gravity direction and active air flow A caused by the fan 250.

In the passive air flow P, the air heated by the heat sink 330 in the body may have a temperature higher than a temperature of the external or surrounding air and may become lighter than such surrounding air, so that the heated air rises upward. The passive air flow P may not be the air flow by the external force but circulation of the air according to the natural convection due to the difference in the specific gravity of the air. Meanwhile, in contrast to the passive air flow P, the active air flow A may refer to forced air flow made by the fan 250 in the body 20. As the active air flow A may be induced by the exerted force, the active air flow A may be formed in any directions as desired.

To facilitate the discharge of the air flow, the cooling device, particularly the body 20 thereof may be configured to combine the active air flow A with the passive air flow P while the cooling device in use, i.e., in the inclined posture. For such a combination, the outlet 221 may be configured to be oriented substantially parallel to the ground when the cooling device in use. As the outlet 221 may be formed at the third region 20c, the inwardly inclined portion, the outlet 221 may be oriented substantially parallel to the ground while the cooling device is in the inclined posture. More specifically, to be parallel to the ground, the outlet 211, particularly the third region 20c may be inclined with regard to the center axis of the cooling device within an angle range 30-60 degrees with which the cooling device in use is inclined. As the outlet 221 may be configured to be parallel to the ground while the cooling device in use, the passive air flow P travelling upward, i.e., in a direction normal to the ground may be directly discharged through the outlet 221 with no resistance. In another exemplary use, the cooling device may be inclined such that the inlet 211 locates at a position vertically lower than the outlet 221. With the location of the inlet 211 vertically lower than the outlet 221, the hot air at the heat sink 330 may naturally flow, and may produce the passive air flow P toward outlet 221. Further, the fan 250 may be configured to produce the active air flow A in the same direction as the passive air flow P when the cooling device is in use. More specifically, as described above referring related drawings, the fan 250 may generate the active air flow A from the front portion of the cooling device toward the rear portion with the smoothly curved path. When the cooling device is erected or inclined while in operation, as shown in FIG. 2F, such active air flow A may be oriented or directed upward, and thus may be aligned generally in the same vertical direction as the passive air flow P.

With such a configuration as described above, the passive air flow P may serve as additional force acting in the same direction as the force by the fan 250 and thus may accelerate the air flow within the body. Therefore, the active air flow A and passive air flow P may be combined and proceed in the same direction and thus the cooling of the heat sink 330 may be greatly enhanced. As shown in FIG. 2F, while the cooling device is in use or in operation, with a posture having a predetermined angle to the ground or the target, such a combination of the passive and active air flow P and A may be greatly facilitated resulting in enhancing the cooling of the heat sink 330 and the discharging of the heated air.

Meanwhile, the thermoelectric cooling unit may be provided at the first end portion 210, and the power source 400 and the controller 500 may be disposed at the second end portion 220. The partition 240 may be installed between the thermoelectric cooling unit and the power source 400/the controller 500 in the body 20. In other words, the first and second end portions 210 and 220 may be divided by the partition 240 to separate the thermoelectric cooling unit and the power source 400/the controller 500 from each other. With such a partition 240, the thermoelectric cooling unit and the power source 400/the controller 500 may be physically and thermally isolated from each other. Therefore, any heated air made around the thermoelectric cooling unit may not flow into the power source 400/the controller 500, and thus the power source 400/the controller 500 may correctly operate not affected by the heated air

Although the components of the cooling device related to cooling and heat dissipating are described separately, they may be categorized again as follows for better understanding, in view of their functions.

The thermoelectric cooling unit may comprise the cooling medium 310 thermally coupled with the cooling tip 10, and the thermoelectric element 320 providing the cooling power to the cooling medium 310. The heat dissipation unit may comprise the heat sink 330 dissipating the heat from the thermoelectric element 320 and the fan 250 providing the air flow to the heat sink 330 for cooling.

Cooling Tip and Tip Case Configuration

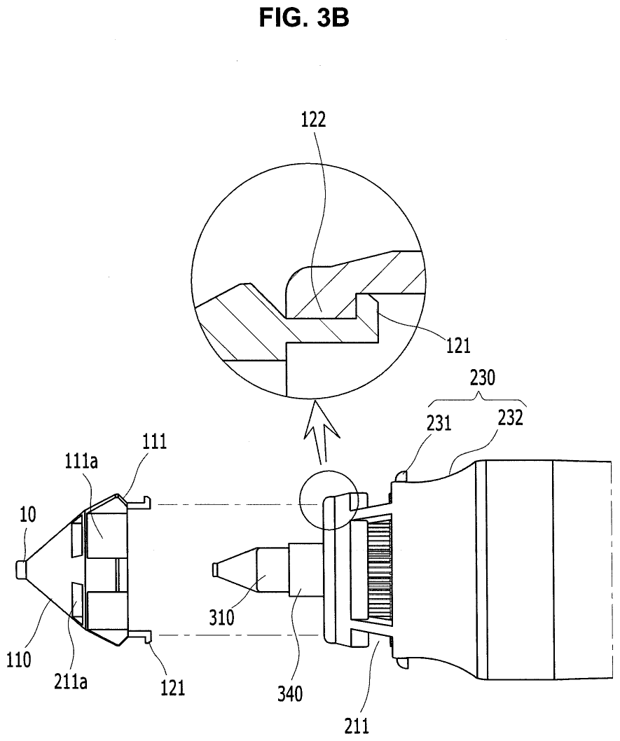

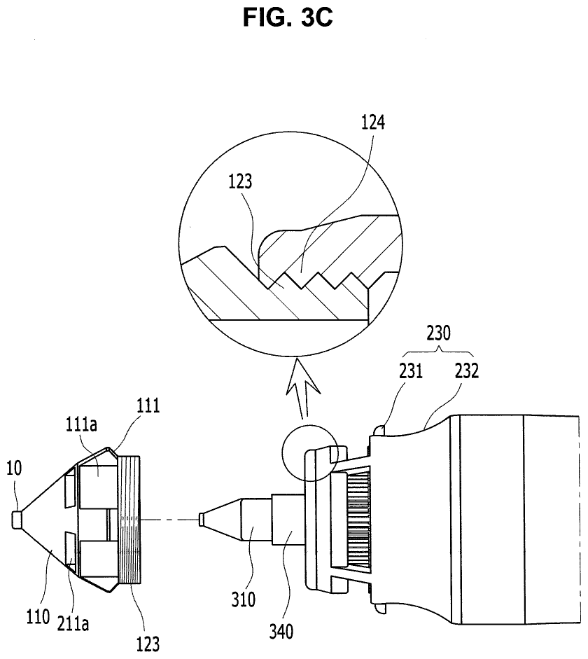

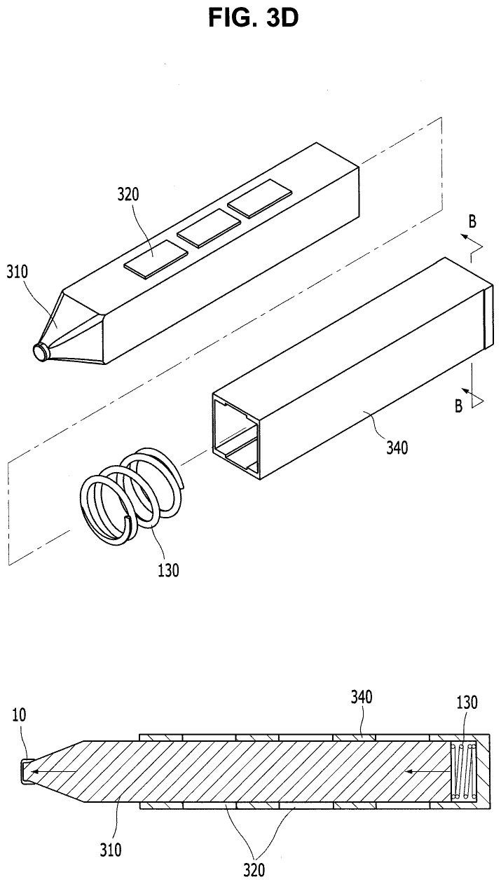

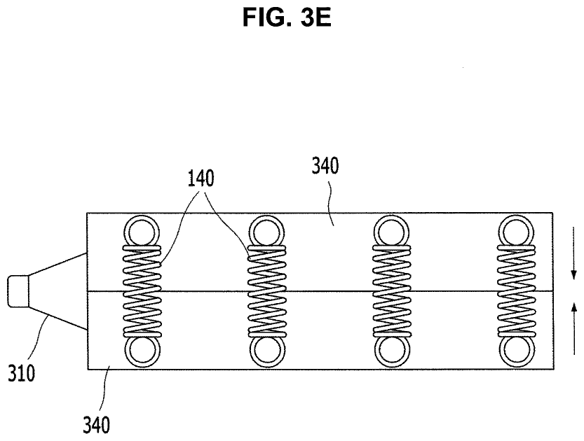

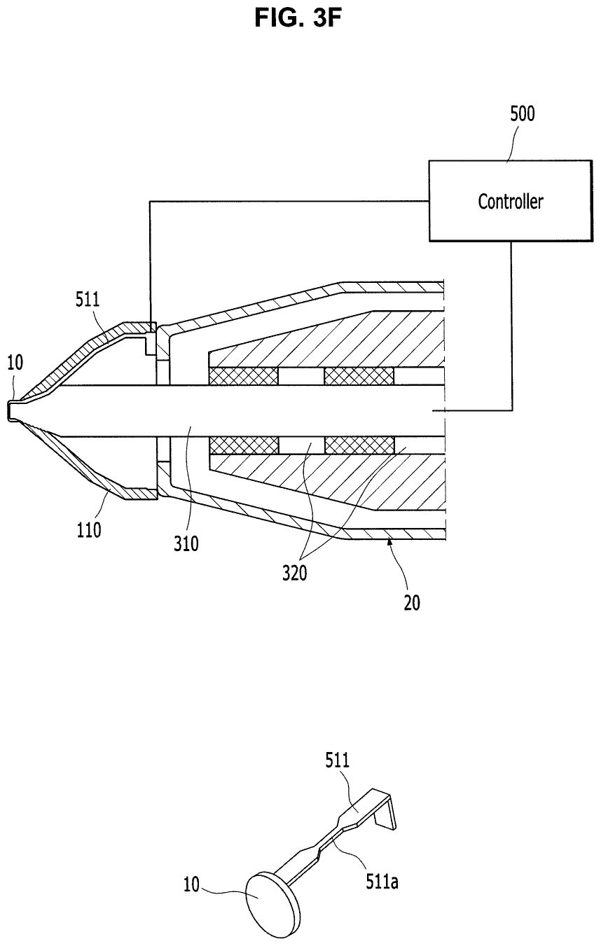

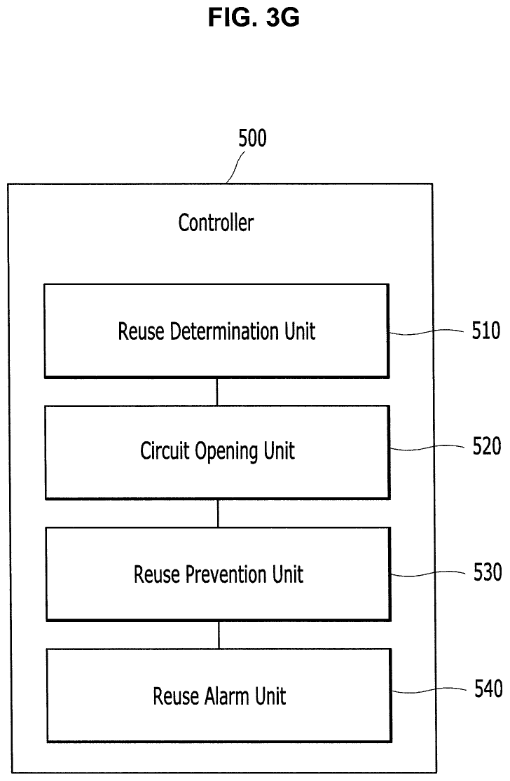

FIGS. 3A-3E are views showing a configuration for the tip case of the cooling device according to the embodiment of the present disclosure.

In the cooling device, the removable cooling tip 10 may be configured to directly contact and cool the target. Further, the tip case 110 may be configured to support the cooling tip 10 and may include a coupling member 120 for attaching the cooling tip 10 to the cooling device. Referring to the related drawings, the cooling tip 10 and the tip case 110 will be described in detail.

FIG. 3A is a view showing a configuration of the removable tip according to the embodiment of the present disclosure.

Referring to FIG. 3A, the electric potential of the cooling tip 10 is stabilized at a certain electric potential. In some embodiments, the cooling tip 10 may have a thermal coupling as well as an electric potential coupling with the cooling medium 310 through a physical and direct contact with the cooling medium 310. The electric potential coupling may be realized by a configuration that the cooling tip 10 has an electric potential corresponding to an electric potential of the cooling medium 310, which is electric-potentially coupled with a component that can store electric charges such as an electric storage or battery.

More specifically, the cooling medium 310 may be physically in contact with the thermoelectric element 320. Further, the cooling medium 310 may be coupled to the removable tip 10 in a physical manner. The cooling medium 310 may also be coated with or made of metal that has flatness better than 100 micrometer and excellent heat transferability. Therefore, due to such a physical coupling, the cooling tip 10 may be adapted to thermally and electric-potentially coupled to the cooling medium 310. Meanwhile, the thermoelectric element 320 may comprise two opposite substrates and the two different conductors interposed between the substrates. These substrates may be made of ceramic or another dielectric material that does not conduct electricity. Further, one of the substrates may actually contact with the cooling medium 310 to provide the cooling power. Since the heat transfer between the thermoelectric element 320 and the cooling medium 310 occurs through lattice vibration, a significant amount of heat within the cooling medium 310 made of metallic materials may propagate due to high electrical conductivity within the cooling medium 310. Accordingly, the cooling power transfer between the cooling medium 310 and the cooling tip 10 is correlated with electric-potential coupling between the cooling medium 310 and the cooling tip 10.

Such coupled potentials between the cooling medium 310 and the cooling tip 10 may be stabilized by the electrical coupling of the cooling tip 10 and/or the cooling medium 310 with an element configured to function as an electric storage. More specifically, the electric charge of the cooling medium 310 may be drained to the electric storage, and thus the potential may be regulated by the potential coupling as described above. In some embodiments, the electric storage element may be replaced with an element that can store electric charges such as the power source 400 that is electrically connected to both the cooling medium 310 and the thermoelectric element 320. In another embodiment, the electric-potential coupling between the cooling medium 310 and the power source 400 may be realized through the thermoelectric element 320. For such configuration of the potential coupling between the cooling medium 310 and the power source 400 through the thermoelectric element 320, the thickness of the electrical insulating substrate of the thermoelectric element 320 may be smaller than 1 mm. When the power source 400 serves as the electric storage and the electric-potential coupling between the power source 400 and the cooling medium 310 is made through the thermoelectric element 320 without any additional electric storage that is directly coupled with the cooling medium 310, such coupled potentials between the cooling medium 310 and the cooling tip 10 may be stabilized within a range of operating electric-potential of the thermoelectric element 320. For more efficient stabilization of potential, the cooling medium 310 and/or the cooling tip 10 may be coupled to a separate electric storage dedicated to the cooling medium 310, and this may also establish a further potential coupling between the cooling tip 10 and the cooling medium 310.

The electric potential coupling of the cooling tip 10, the cooling medium 310 and the element functioning as the electric storage may be established prior to the treatment by the cooling device, and thus may stabilize the potential in advance well before the contact of the cooling tip 10 with the target. In some embodiments, the potential coupling of the cooling tip 10, the cooling medium 310 and the element functioning as the electric storage may be achieved prior to the treatment and then may be maintained during the treatment.

For these reasons, the cooling tip 10 may have electrical stability and may prevent any electric leakage to the target in the eye while in contact therewith.

Meanwhile, the removable tip 10 may have two functions. According to one embodiment, the removable tip 10 may have a heat transfer function for rapidly cooling the target using the cooling medium 310, which receives the cooling power from the thermoelectric element 320 and cools the removable tip 10 based on the thermal coupling between the tip 10, the medium 310, and the thermoelectric element 320. According to the other embodiment, the removable tip 10 may also have a reuse prevention function for maintaining sterility of the target.

To achieve these functions, particularly the heat transfer function, the cooling tip 10 and the tip case 110 may first need to be coupled to the cooling medium 310 with minimum thermal resistance. In an aspect of the heat transfer, main thermal resistance may be physical clearance between the cooling medium 310 and the cooling tip 10. A fastening mechanism may be required to minimize or remove the physical clearance such that the cooling tip 10 is thermally integrated with the cooling member 310 as one body. As an example according to the present disclosure, FIGS. 2D and 3F show a screw-fastening mechanism. More specifically, a thread portion 311 may be formed at the end or end portion of the cooling medium 310 facing the tip case 110. Further, the cooling tip case 110 may have a threaded portion 112 that is formed on an inner surface thereof and is mated with the thread portion 311 of the cooling medium 310. The cooling medium 310 may be screwed on the tip case 110 using the threaded portions 311 and 112 with being inserted into the tip case 110. The cooling medium 310 may be pushed against the cooling tip 10 while being screwed on. Thus, the cooling medium 310 may closely adhere to the cooling tip 10 with no clearance and may achieve the tightly coupling with no thermal resistance.

FIGS. 3B and 3C shows a configuration for coupling the tip case to the cooling device.

Referring to FIGS. 3B and 3C, the coupling member 120, i.e., the coupling mechanism for the tip case 110 may adopt one of a hook lock mechanism and a screw mechanism. More specifically, in the hook lock mechanism as shown in FIG. 3B, the tip case 110 may include a hook 121 extending toward the body 20 of the cooling device. Further, the body 20 may include a step or a flange 122 formed at the end of the body 20 and extending inwardly in a radial direction. The tip case 110 may be coupled to or decoupled from the body 20 by latching the hook 121 on the flange 122 or releasing the hook 121 from the flange 122. In the screw mechanism as shown in FIG. 3C, a thread portion 123 may be formed at a rear end portion of the cooling tip case 110 facing the body 20. Further, the body 20 may have a threaded portion 124 that is formed on an inner surface of the end of the body 20 and is mated with the thread portion 123 of the tip case 110. The tip case 110 may be screwed on the body 20 using the threaded portions 123 and 124. When the tip case 110 is coupled to the body using these mechanisms, the cooling tip 10 in the tip case 110 may be thermally coupled to the cooling medium 310 protruding from the body 20 via the physical contact therewith such that the cooling tip 10 may receive the cooling power from the cooling member 310 and cools the target.

The tip case 110 may include a grip 111 formed adjacent to the rear end thereof that is coupled to the body 20 of the cooling device. The grip 111 may provide a space for the finger to manipulate the tip case 110 while the tip case 110 is attached to or detached from the cooling device. For example, the grip 111 may be formed inclined inwardly toward the center axis of the cooling device. That is, the grip 111 may comprise a tapered portion of the tip case 110 extending to adjoin the front end of the body 20 of the cooling device. Such a grip 111 may define a surface for rigidly supporting the fingers and thus the tip case 110 may be easily attached to or detached form the cooling device using the grip 111.

Additionally, the tip case 110 may include a sub grip 111a formed on an outer surface of the tip case body to facilitate attaching or detaching of the tip case 110. The sub grip 111a is also well shown in FIGS. 1D and 2B. The sub grip 111a may provide a space receiving the finger while the tip case 110 is attached to or detached from the body 20 of the cooling device. The sub grip 111a may comprise a recess formed on the body of the tip case 110 to define the space for the finger. A plurality of sub grips 111a may be provided along a circumferential direction of the tip case 110 with a predetermined distance. The user may place the fingers in the sub grips 111a and may easily rotate the tips case 110 to be attached to or detached from the body 20.

In view of the configuration as described above, the grip 111 may be advantageous for pressing the tip case 110 and thus may be adapted to be used along with the hook lock mechanism as shown in FIG. 3B. In contrast, the sub grip 111a may be advantageous for rotating or twisting the tip case 110 and thus may be adapted to be used along with the screw mechanism as shown in FIG. 3C

In some embodiments of the present disclosure, the cooling device may have a pressing mechanism configured to apply pressure on the cooling medium 310 to achieve a close adhesion to the cooling tip 10. Such a pressing mechanism will be described as follows. FIGS. 3D and 3E are views showing configurations of the cooling unit having elastic members according to the embodiment of the present disclosure.

In one embodiment, the cooling medium 310 may be immovably installed within the containers 340. However, in another embodiment, the cooling medium 310 may be configured to be detachably coupled to the container 340. That is, the cooling medium 310 may be movable within the container 340. In this case, the coupling between the cooling medium 310 and the cooling tip 10 may be enhanced by applying an elastic member 130 to the cooling medium 310. Referring to FIG. 3D, the elastic member 130 may be configured to apply elastic force to the cooling medium 310 toward the cooling tip 10. More specifically, the elastic member 130 may be provided between a rear end of the cooling member 310 and a rear end of the container 340. The elastic member 130 may be configured to support the cooling member 310 and push the cooling member 310 against the cooling tip 10 while being deformed between the cooling medium 310 and the container 340. Therefore, the cooling member 310 may closely contact the cooling tip 10 for improving the thermal coupling with the cooling tip 10.

In some embodiments, the physical and thermal coupling between the cooling tip 10 and the cooling medium 310 may be facilitated by a smooth interface. Specifically, the roughness of the surfaces that form the interface between the cooling tip 10 and the cooling medium 310 may be less than 5 μm Ra. In another embodiment, a thin medium layer having thermal conductivity larger than 1 W/m-K such as thermal paste and graphite film may be used at the interface between the cooling tip 10 and the cooling medium 310.

Referring to FIG. 3E, the cooling device may further include an elastic member 140 configured to apply the elastic force to the containers 340. More specifically, the elastic member 140 may elastically connect the pair of containers 340. The elastic member 140 may be coupled to sides of these containers 340 and may extend in a lateral direction over these containers 340. When the cooling medium 310 are coupled to the containers 340, the elastic member 140 may relatively press the containers 340 against the cooling medium 310 while being deformed. Therefore, using the elastic member 140, the cooling medium 310 may be securely accommodated in the containers 340.

FIG. 3F is a view showing a configuration of the tip case according to the embodiment of the present disclosure and FIG. 3G is a view showing a logical structure or circuitry of the controller according to the present disclosure.

Referring to FIG. 3F, the tip case 110 may include a body and a connector 511 disposed in the body. The connector 511 may be made of material with the electrical conductivity and may comprise a narrow strip. The connector 511 may extend within the body of the tip case 110 from the cooling tip 10 to the rear end of the body of tip case 110 that is coupled to the body 20. The connector 511 may be placed on the inner surface of the tip case 110 to be supported thereby and may extend along the inner surface of the tip case 110. One end of the connector 511 may be connected with the cooling tip 10 and the other end of the connector 511 may be connected with the controller 500 via any electrically conductive member interposed between the connector 511 and the controller 500 such as a metal wire. Such a wire may extend from the connector 511 through the body 20 to the controller 500. Thus, the connector 511 may electrically connect the cooling tip 10 and the controller 500.

Further, as shown in FIG. 3F, the cooling medium 310 may be also electrically connected with the controller 500 via any electrical conductive member. The cooling medium 310 may be electrically coupled directly with the controller 500. Alternatively, the cooling medium 310 may be electrically coupled with the controller 500 via the thermoelectric element 320 which is controlled by the controller 500 with being electrically connected to the controller 500. Therefore, when the cooling medium 310 is coupled to the cooling tip 10, the cooling medium 310, the cooling tip 10, and the connector 511 may form a closed electric circuit along with the controller 500. Using such a circuit, the controller 500 may confirm whether the cooling tip 10 is coupled. That is, if controller 500 receives an electric signal transmitted from and returned to the controller 500 through the circuit as described above, the controller 500 may determine that the circuit is closed and the cooling tip 10 is coupled to the cooling medium 310. If not, the controller 500 may determine that the circuit is opened and the cooling tip 10 is not coupled to the cooling medium 310. Once the cooling tip 10 is coupled to the cooling medium 310, the controller 500 may recognize such a coupling based on the returned signal though the circuit. In the similar manner, the controller 500 may recognize the decoupling of the cooling tip 10 by failing to receive the returned signal.

Further, the controller 500 may determine whether the cooling tip 10 is reused or not, using the circuit. When the cooling tip 10 is coupled, the controller 500 may receive the signal from the established closed circuit and such a signal may acts as a count and stored in the controller 500. If the cooling tip 10 is decoupled and such a decoupling is recognized by the controller 500, the controller 500 may reset the count of the use. The controller 500 may be configured to transmit to and receive from the circuit to check the coupling of the cooling tip 10 every time the cooling device is in use or operation to cool the target. If the cooling tip 10 previously used is not decoupled and used again, the controller 500 may add to the count when receiving the returned signal and may determine that total count of use is greater than the predetermined count, i.e., a single time. In this case, the controller may disable or inactivate the cooling device and thus the reuse of the cooling tip 10 may be prevented. Further, the controller 500 may provide an indication to the user in order to prevent the reuse. For example, the indication may be beeping or lighting.

Meanwhile, using the circuit formed between the tip 10, the medium 310, and the connector 511, the controller 500 may be further configured to determine the reuse of the cooling tip 10 in a different manner, i.e., based on unavailability of the connector 511. More specifically, as described above, the connector 511 may be thin and narrow member and may be in contact with the body of the tip case 110. When the tip case 110 is pressed or twisted to be detached from the body 20 of the cooling device, the external force acts on the connector and thus the connector 511 may be deformed or broken down. Due to the deformation, the connector 511 may not be connected to the controller 511 via the wire, and the circuit may become opened even though the tip case 110 with the cooling tip is coupled to the body 20 again. Therefore, the controller 500 may not receive any returned signal from the circuit and thus may determine that the cooling tip 10 currently being coupled has been used. In this case, the controller 500 may interrupt the activation of the cooling device, and thus the reuse of the cooling tip 10 may be restricted. Alternatively, if the controller 500 receives any returned signal from the closed circuit, the controller 500 may determine that the cooling tip 10 has not been used and is in normal use. In this case, the controller 500 may normally operate the cooling device to proceed with cooling the target.

Alternatively, in view of the thin and narrow body thereof, the connector 511 may be blown out like a fuse by an excessive electric current. Further, the connector 511 may comprise a separate fuse 511a including a portion of the connector thinner and narrower than other portions of the connector 511. After the single use of the cooling tip 10, the controller 500 may apply the excessive current to the connector 511 to be blown out. As already mentioned above, the circuit may become opened even though the tip case 110 along with the cooling tip 10 is coupled to the body 20 again. Accordingly, the controller 500 may interrupt the activation of the cooling device to prevent the reuse of the cooling tip 10 since the controller 500 fails to receive any returned signal.

As described above, the unavailability of the connector 511, i.e., the deformation or the breakage thereof may be directly associated with the opening of the electric circuit, which then results in the reuse of the cooling tip 10. Therefore, in the above configuration for detecting the reuse, the connector 511 may be regarded as the electric circuit itself configured to determine the reuse of the cooling tip 10.

As shown in a perspective view of FIG. 3F, the cooling tip 10 may be assembled with the connector 511 as one body to improve productivity. In this case, the cooling tip 10 may correspond to a head of such an assembly and the connector 511 may correspond to a leg thereof. As the configurations of the head 10 and leg 511 are described above in detail with reference to the tip 10 and the connector 511, further description will be omitted.

As described above, the electric circuit may be configured to be opened in an electrical manner using the excessive current or in a physical manner using the force exerted on the tip case 110. Further, at least one of the cooling tip 10 and the cooling medium 310 may be grounded through an electrical connection with the cooling device, i.e., the potential coupling.

In view of the operation and the function as describe above, the controller may be divided into functional units or circuitries, as shown in FIG. 3G. The controller 500 may include a reuse determination unit 510 for determining whether the cooling tip 10 is reused or normally used by detecting the electrical connection through the circuit, and a reuse prevention unit 530 configured to limit use of the cooling device when it is determined that the cooling tip 10 is reused by the determination unit 510. Further, the controller 500 may comprise a circuit opening unit 520 for opening the electric circuit for preventing reuse, if the reuse determination unit 510 determines that the cooling tip is in the normal use, i.e. the first use. The electrical circuit may be opened by the deformation of the connector 511 caused by the force acting on the connector 511 through the tip case 110. Alternatively, the electric circuit may be opened by breaking or cutting the connector 511 using the excessive current. In this case, the circuit opening unit 520 may be configured to apply the excessive current to the connector 511 if the determination unit 510 determines that the cooling tip 10 is in normal use. The controller may further comprise a reuse alarm unit 540 signaling the reuse of the cooling tip 10. The reuse alarm unit 540 may be configured to use one or more of display, sound, or vibration to inform the user of the reuse of the cooling tip 10. The controller 500, specifically the determination unit 510 may determine whether the cooling tip 10 is reused when the cooling tip 10 is coupled with the cooling medium 310, and may transmit a result of determination as the electric signal to the reuse alarm unit 540.

The cooling device and method according to the present disclosure may have the following advantages.

The cooling device and method according to the embodiment of the present disclosure may solve severe patient's waiting and clinic work load due to time-consuming anesthesia required by conventional therapies and may significantly reduce psychological burden and pain of the patients with ocular anesthetic agents.

According to some embodiments, the user may treat the patient while stably gripping the cooling device through a configuration considering center of mass and ergonomics.

According to some embodiments, contamination or contagion via the cooling device may be effectively prevented by adopting a disposable cooling tip.

According to some embodiments, the cooling device is stably operated while rapidly anesthetizing the target based on a configuration facilitating internal air flow for effective heat dissipation.

Although embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. A device for cooling a living tissue comprising:
a body including a first end portion and a second end portion positioned opposite to the first end portion;
a cooling unit disposed at the first end portion and including a cooling medium configured to cool a target area;
a heat dissipating unit disposed at the first end portion and dissipating heat from the cooling unit, the heat dissipating unit further including a fan for generating an active air flow;
a partition disposed between the first end portion and the second end portion, the partition being configured to isolate the first end portion from the second end portion; and
at least one inlet introducing external air from an outside of the device and at least one outlet discharging internal air in the device, the at least one inlet and the at least one outlet being provided at a predetermined region of the first end portion isolated from the second end portion by the partition, such that air flow between the at least one inlet and the at least one outlet is formed within the predetermined region of the first end portion,
wherein the partition is configured to redirect air flow oriented toward the second end portion to the at least one outlet, and
wherein air flow discharged at the at least one outlet comprises a first air flow discharged directly from the fan and a second air flow redirected by the partition, and the at least one inlet and the at least one outlet are disposed on a region having a predetermined inclination angle in the body.
